(19) United States
(12) Patent Application Publication
(10) Pub. No.: US 2024/0138750 A1
(43) Pub. Date: May 2, 2024

(54) COGNITIVE FUNCTION ESTIMATION DEVICE, COGNITIVE FUNCTION ESTIMATION METHOD, AND STORAGE MEDIUM

(71) Applicant: NEC Corporation, Minatoku, Tokyo (JP)

(72) Inventors: Terumi Umematsu, Tokyo (JP); Masanori Tsujikawa, Tokyo (JP)

(73) Assignee: NEC Corporation, Minatoku, Tokyo (JP)

(21) Appl. No.: 18/279,135

(22) PCT Filed: Jun. 29, 2021
(86) PCT No.: PCT/JP2021/024506

§ 371 Date: Aug. 28, 2023

(51) Int. Cl. A61B 5/00; G16H 10/60; G16H 20/30; G16H 50/30

(52) U.S. Cl. CPC A61B 5/4088; G16H 10/60; G16H 20/30; G16H 50/30

(57) ABSTRACT

A cognitive function estimation device 1X mainly includes a first state information acquisition means 15X, a second state information acquisition means 16X, and a cognitive function estimation means 17X. The first state information acquisition means 15X is configured to acquire first state information representing a first state of a subject regarding a cognitive function of the subject. The second state information acquisition means 16X is configured to acquire second state information representing a second state of the subject whose interval of state change is longer than the first state. The cognitive function estimation means 17X is configured to estimate the cognitive function of the subject based on the first state information and the second state information.

### TECHNICAL FIELD

The present disclosure relates to a technical field of a cognitive function estimation device, a cognitive function estimation method, and a storage medium configured to perform processing related to estimation of a cognitive function.

### BACKGROUND

There is a device or a system configured to estimate the cognitive function of a subject. For example, Patent Literature 1 discloses a cognitive function measurement device that calculates an evaluation value regarding a cognitive function based on gait data of a subject. Further, Non-Patent Literature 1 discloses a technique of examining the cognitive function of a subject based on the facial data (especially measurement information regarding the line of sight) of the subject. Further, Non-Patent Literature 2 discloses a technique of determining whether or not a subject is a major neurocognitive disorder from a face image of the subject using a deep learning-based model. Non-Patent Literature 3 discloses measurement results, through comparison of gaits between a person with Alzheimer dementia and a person with Lewy body dementia, indicating that the asymmetry of the step time and the swing phase of a subject with Lewy body dementia is more remarkable and the variance of the step time and the step length of a person with Lewy body dementia is larger than that of a person with Alzheimer dementia. In general, it is known that a person with late Alzheimer dementia has gait tendencies of slow walk and a head forward posture and a lateral inclination posture. In contrast, it is known that a person with late Lewy body dementia has gait tendencies of a shuffle, a head forward posture, and a small arm swing. It is known that a person with vascular dementia has gait tendencies of a short step gait, a large step gait, and a shuffling gait.

### CITATION LIST

### Patent Literature

Patent Literature 1: WO2021/075061

### NON-PATENT LITERATURE

Non-Patent Literature 1: Akane Oyama, et al. “Novel Method for Rapid Assessment of Cognitive Impairment Using High-Performance Eye-Tracking Technology”, Scientific reports 9(1) 12932, 2019.
Non-Patent Literature 2: Yumi Umeda-Kameyama, et al. “Screening of Alzheimer's disease by facial complexion using artificial intelligence”, Aging, Research Paper, Volume 13, Issue 2, pp 1765-1772, 2021.
Non-Patent Literature 3: Riona Mc Ardle, et al. “Do Alzheimer's and Lewy body disease have discrete pathological signatures of gait?”, ELSEVIER, Alzheimer's & Dementia 1-11, 2019.

### SUMMARY

### Problem to be Solved

For the purpose of increasing health expectancy in the aging society, there is a growing demand for early detection of a decline in the cognitive function. In addition, the decline in the cognitive function occurs not only in the elderly people but also in people in the working generation. In the latter case, perceiving the decline in the cognitive function is difficult and therefore it is difficult to notice it. Therefore, in addition to the measurement of cognitive function by examination in a medical institution, it is conceivable to estimate the cognitive function conveniently in daily life. However, there is an issue that the estimation accuracy deteriorates, if the estimation of the cognitive function is carried out by a method simpler than the examination in a medical institution.

In view of the above-described issue, it is therefore an example object of the present disclosure to provide a cognitive function estimation device, a cognitive function estimation method, and a storage medium capable of accurately estimating a cognitive function.

### Means for Solving the Problem

In one mode of the cognitive function estimation device, there is provided a cognitive function estimation including:
a first state information acquisition means configured to acquire first state information representing a first state of a subject regarding a cognitive function of the subject;
a second state information acquisition means configured to acquire second state information representing a second state of the subject whose interval of state change is longer than the first state; and
a cognitive function estimation means configured to estimate the cognitive function of the subject based on the first state information and the second state information.

In another mode of the cognitive function estimation device, there is provided a cognitive function estimation including:
an acquisition means configured to acquire
   facial data which is measurement information regarding a face of a subject and
   gait data which is measurement information regarding a gait state of the subject; and
a cognitive function estimation mean configured to estimate a cognitive function of the subject based on the facial data and the gait data.

In one mode of the cognitive function estimation method, there is provided a cognitive function estimation method executed by a computer, the cognitive function estimation method including:
acquiring first state information representing a first state of a subject regarding a cognitive function of the subject;
acquiring second state information representing a second state of the subject whose interval of state change is longer than the first state; and
estimating the cognitive function of the subject based on the first state information and the second state information.

In another mode of the cognitive function estimation method, there is provided a cognitive function estimation method executed by a computer, the cognitive function estimation method including:
acquiring
   facial data which is measurement information regarding a face of a subject and
   gait data which is measurement information regarding a gait state of the subject; and
estimating a cognitive function of the subject based on the facial data and the gait data.

In one mode of the storage medium, there is provided a storage medium storing a program executed by a computer, the program causing the computer to
acquire first state information representing a first state of a subject regarding a cognitive function of the subject;
acquire second state information representing a second state of the subject whose interval of state change is longer than the first state; and
estimate the cognitive function of the subject based on the first state information and the second state information.

In another mode of the storage medium, there is provided a storage medium storing a program executed by a computer, the program causing the computer to
acquire
   facial data which is measurement information regarding a face of a subject and
   gait data which is measurement information regarding a gait state of the subject; and
estimate a cognitive function of the subject based on the facial data and the gait data.

### Effect

An example advantage according to the present invention is to accurately estimate a cognitive function.

### BRIEF DESCRIPTION OF THE DRAWINGS

FIG. 1 It shows a schematic configuration of a cognitive function estimation system according to a first example embodiment.

FIG. 2 It shows a hardware configuration of an information processing device.

FIG. 3 It is a diagram schematically showing elements that affect the cognitive function.

FIG. 4 It is an example of functional blocks of the information processing device.

FIG. 5 It is a diagram showing a specific example of the estimation of the cognitive function.

FIG. 6 It is an example of functional blocks of a cognitive function estimation device regarding the learning of the inference model.

FIG. 7 It is an example of a flowchart showing a processing procedure related to the estimation of cognitive function.

FIG. 8 It shows a schematic configuration of a cognitive function estimation system according to a second example embodiment.

FIG. 9 It is a block diagram of the cognitive function estimation device according to a third example embodiment.

FIG. 10 It is an example of a flowchart executed by the cognitive function estimation device according to the third example embodiment.

FIG. 11 It is a block diagram of a cognitive function estimation device according to a fourth example embodiment.

FIG. 12 It is an example of a flowchart executed by the cognitive function estimation device according to the fourth example embodiment.

### EXAMPLE EMBODIMENTS

Hereinafter, example embodiments of a cognitive function estimation device, a cognitive function estimation method, and a storage medium will be described with reference to the drawings.

### First Example Embodiment

(1) System Configuration

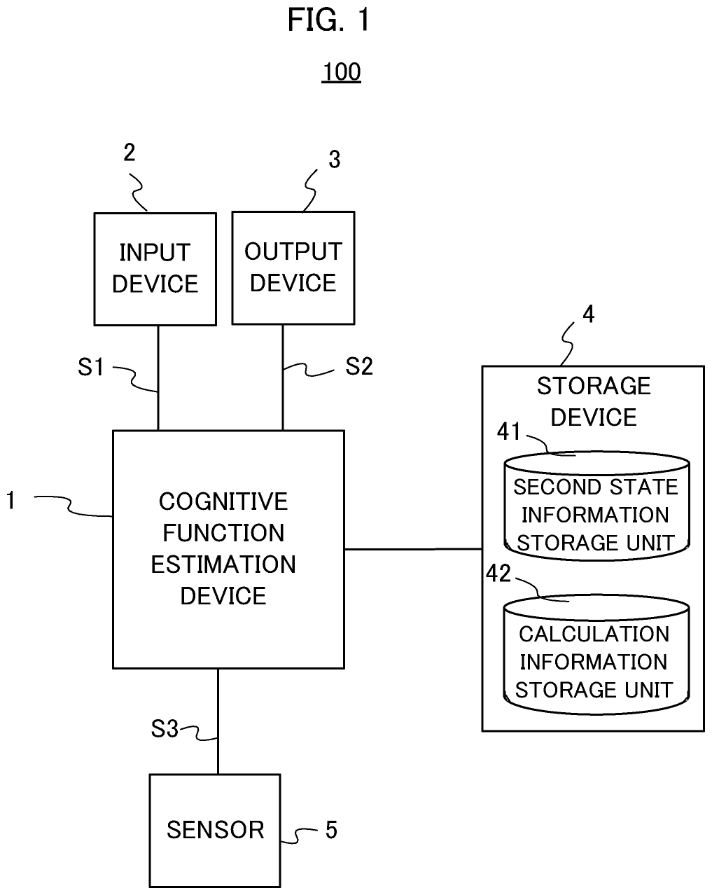

FIG. 1 shows a schematic configuration of a cognitive function estimation system 100 according to a first example embodiment. The cognitive function estimation system 100 estimates the cognitive function of a subject with high accuracy without giving an excessive measurement load to the subject to present the estimation result. Here, the “subject” may be a target person of management of the cognitive function by an organization, or may be an individual user.

The cognitive function estimation system 100 mainly includes a cognitive function estimation device 1, an input device 2, an output device 3, a storage device 4, and a sensor 5.

The cognitive function estimation device 1 performs data communication with the input device 2, the output device 3, and the sensor 5 through a communication network or through wireless or wired direct communication. The cognitive function estimation device 1 estimates the cognitive function of a subject based on an input signal “S1” supplied from the input device 2, a sensor (detection) signal “S3” supplied from the sensor 5, and information stored in the storage device 4. In this case, the cognitive function estimation device 1 estimates the cognitive function of the subject with high accuracy by considering not only a state (also referred to as “first state”) that is a temporary state (a state which varies in the short term) of the subject but also a state (also referred to as “second state”) of the subject that varies at an interval longer than the interval of the first state. In this case, for example, the cognitive function estimation device 1 calculates a score (in the case of MMSE, on a scale of 30 points) of the cognitive function to be adopted in any neuropsychological examination such as MMSE (Mini-Mental State Examination), as the estimation result of the cognitive function. Hereafter, as an example, the explanation is made on the assumption that the higher the above score is, the higher the cognitive function becomes (i.e., closer to normal). The cognitive function estimation device 1 generates an output signal “S2” regarding the estimation result of the cognitive function of the subject and supplies the generated output signal S2 to the output device 3.

The input device 2 is one or more interfaces that receive manual input (external input) of information regarding each subject. The user who inputs the information using the input device 2 may be the subject itself, or may be a person who manages or supervises the activity of the subject. The input device 2 may be a variety of user input interfaces such as, for example, a touch panel, a button, a keyboard, a mouse, and a voice input device. The input device 2 supplies the generated input signal S1 to the cognitive function estimation device 1. The output device 3 displays or outputs predetermined information based on the output signal S2 supplied from the cognitive function estimation device 1. Examples of the output device 3 include a display, a projector, and a speaker.

The sensor 5 measures a biological signal regarding the subject and supplies the measured biological signal to the cognitive function estimation device 1 as a sensor signal S3. In this instance, the sensor signal S3 may be any biological signal (including vital information) regarding the subject such as a heart rate, EEG, pulse wave, sweating volume (skin electrical activity), amount of hormonal secretion, cerebral blood flow, blood pressure, body temperature, myoelectric potential, respiration rate, and acceleration. The sensor 5 may also be a device that analyzes blood collected from the subject and outputs a sensor signal S3 indicative of the analysis result. Examples of the sensor 5 include a wearable terminal worn by the subject, a camera for photographing the subject, a microphone for generating a voice signal of the subject's utterance, and a terminal such as a personal computer or a smartphone operated by the subject. For example, the above-described wearable terminal includes a GNSS (global navigation satellite system) receiver, an acceleration sensor, a sensor for detecting biological signals, and the like, and outputs the output signals from each sensor as a sensor signal S3. The sensor 5 may supply information corresponding to the manipulation amount signal from a personal computer or a smartphone to the cognitive function estimation device 1 as the sensor signal S3. The sensor 5 may also output a sensor signal S3 representing biomedical data (including sleep time) regarding the subject during the sleep.

The storage device 4 is a memory for storing various information necessary for processing performed by the cognitive function estimation device 1. The storage device 4 may be an external storage device, such as a hard disk, connected to or embedded in the cognitive function estimation device 1, or may be a storage medium, such as a flash memory. The storage device 4 may be a server device that performs data communication with the cognitive function estimation device 1. Further, the storage device 4 may be configured by a plurality of devices.

The storage device 4 functionally includes a second state information storage unit 41 and a calculation information storage unit 42.

The second state information storage unit 41 stores the second state information which is information regarding the second state of the subject. Here, examples of the second state information include: disorder information (including the diagnosis result by a doctor) regarding the disorder (illness) of the subject; life habit information regarding a life habit of the subject; genetic information; and attribute information regarding various characteristics (including the age, race, gender, occupation, hobby, preference, or/and personality) of the subject.

The second state information may be data converted to be data which conforms to the input format to a model, wherein the model is used by the cognitive function estimation device 1 in the cognitive function estimation to be described later. In this case, the second state information is data obtained through feature extraction process to the above-mentioned disorder information, the life habit information, and/or the attribute information, and the like and is expressed in a predetermined tensor format (e.g., feature vector). This feature extraction process may be process based on an arbitrary feature extraction technique (including a feature extraction technique based on deep learning using a neural network or the like). The generation of the second state information is performed before the estimation of the cognitive function, or may be performed by the cognitive function estimation device 1, or may be performed by a device other than the cognitive function estimation device 1.

Here, a supplementary description will be given of a method of generating the second state information. According to a first example, the second state information is generated based on the questionnaire result. For example, there is Big Five Personality Test as a questionnaire for judging the personality. In addition, there are questionnaires regarding a life habit. The individual attribute information such as age, gender, job type, and race may also be generated as an answer of a questionnaire. According to a second example, the second state information is generated by an image recognition technique (e.g., a technique to generate age information or human race information regarding a person included in an image) using an image obtained by photographing a subject. According to a third example, the second state information may be information based on the measurement results of the first state, which is a temporary state of the subject, measured continuously for a predetermined period of time (e.g., one month or more). In the third example, for example, statistical data obtained by applying an arbitrary statistical analysis process to the time-series measurement results of the first state of the subject continuously measured for a predetermined period of time is stored in the second state information storage unit 41 as the second state information. The second state information generated in the third example corresponds to the life habit information regarding the subject.

The calculation information storage unit 42 stores calculation information that is information to be used for calculation of the estimation result (score) of the cognitive function. The calculation information is information regarding a model configured to calculate a score of the cognitive function of the subject from the first state information that is information regarding the first state of the subject and the second state information.

According to a first example of the calculation information, the calculation information includes: the inference model information regarding an inference model configured to calculate a temporal score of the cognitive function of the subject from the first state information; and correction model information regarding a correction model configured to correct the above-described temporal score on the basis of the second state information. In this first example, the score after correction by the correction model of the temporal score calculated by the inference model is used as the final estimation result (score) of the cognitive function. The correction model in this first example may be a model which determines the correction amount of the temporal score to vary continuously or stepwise in accordance with the second state information. In this case, for example, the correction model may be a look-up table showing a combination of second state information to be assumed and the correction amount to be applied according thereto, or may be an expression or any other calculation model for calculating the correction amount from the second state information. In yet another example, the correction model may be a model configured to calculate the score of the cognitive function from the second state information and the temporal score. If the second condition information is classified based on whether or not it has a good influence on the cognitive function, the correction model may be a model configured to increase the temporal score by a predetermined value or a predetermined rate if the classification result indicates that the second state information has a good influence, and decrease the temporal score by a predetermined value or a predetermined rate if the classification result indicates that the second state information has a bad influence.

In the second example of the calculation information, the calculation information may include inference model information regarding an inference model trained to output the estimated score of the cognitive function using both the first condition information and the second state information as input data.

Here, the inference model according to the first example or the second example is, for example, a regression model (statistical model) or a machine learning model, and in this case, the calculation information includes information indicative of parameters necessary to build (configure) the above-described model. For example, if the model described above is a model based on a neural network such as a convolutional neural network, the calculation information includes information indicative of various parameters regarding the layer structure, neuron structure of each layer, the number of filters and filter size in each layer, and weight for each element of each filter. The inference model in the second example may be an expression or a look-up table for directly determining the estimated score of the cognitive function from the first state information and the second state information. Similarly, the inference model in the first example (i.e., the model configured to output the temporal score from the first state information) may be an expression or a look-up table for directly determining the estimated score of the cognitive function from the first state information.

The configuration of the cognitive function estimation system 100 shown in FIG. 1 is an example, and various changes may be made to the configuration. For example, the input device 2 and the output device 3 may be configured integrally. In this case, the input device 2 and the output device 3 may be configured as a tablet-type terminal that is incorporated into or separate from the cognitive function estimation device 1. Further, the input device 2 and the sensor 5 may be configured integrally. Further, the cognitive function estimation device 1 may be configured by a plurality of devices. In this case, the plurality of devices constituting the cognitive function estimation device 1 transmits and receives information necessary for executing the preassigned process among the plurality of devices. In this case, the cognitive function estimation device 1 functions as a system.

(2) Hardware Configuration

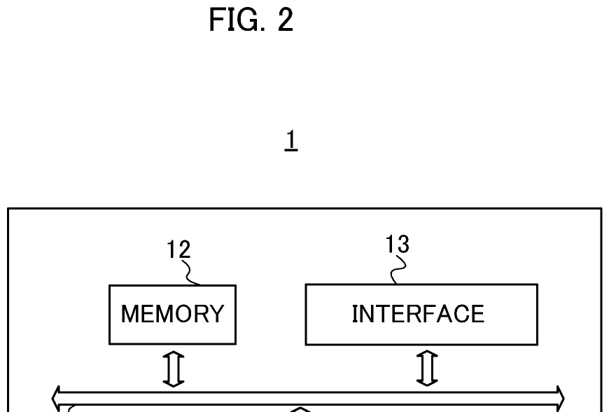

FIG. 2 shows a hardware configuration of the cognitive function estimation device 1. The cognitive function estimator 1 includes a processor 11, a memory 12, and an interface 13 as hardware. The processor 11, memory 12 and interface 13 are connected to one another via a data bus 10.

The processor 11 functions as a controller (arithmetic unit) that performs overall control of the cognitive function estimation device 1 by executing a program stored in the memory 12. Examples of the processor 11 include a CPU (Central Processing Unit), a GPU (Graphics Processing Unit), and a TPU (Tensor Processing Unit). The processor 11 may be configured by a plurality of processors. The processor 11 is an example of a computer.

The memory 12 comprises a variety of volatile and non-volatile memories, such as a RAM (Random Access Memory), a ROM (Read Only Memory), and a flash memory. The memory 12 stores a program for the cognitive function estimation device 1 to execute a process. A part of the information stored in the memory 12 may be stored in one or more external storage devices capable of communicating with the cognitive function estimation device 1, or may be stored in a storage medium detachable from the cognitive function estimation device 1.

The interface 13 is one or more interfaces for electrically connecting the cognitive function estimation device 1 to other devices. Examples of the interfaces include a wireless interface, such as network adapters, for transmitting and receiving data to and from other devices wirelessly, and a hardware interface, such as a cable, for connecting to other devices.

The hardware configuration of the cognitive function estimation device 1 is not limited to the configuration shown in FIG. 2. For example, the cognitive function estimation device 1 may include at least one of the input device 2 and the output device 3. Further, the cognitive function estimation device 1 may be connected to or incorporate a sound output device such as a speaker.

(3) Specific Examples of First State and Second State

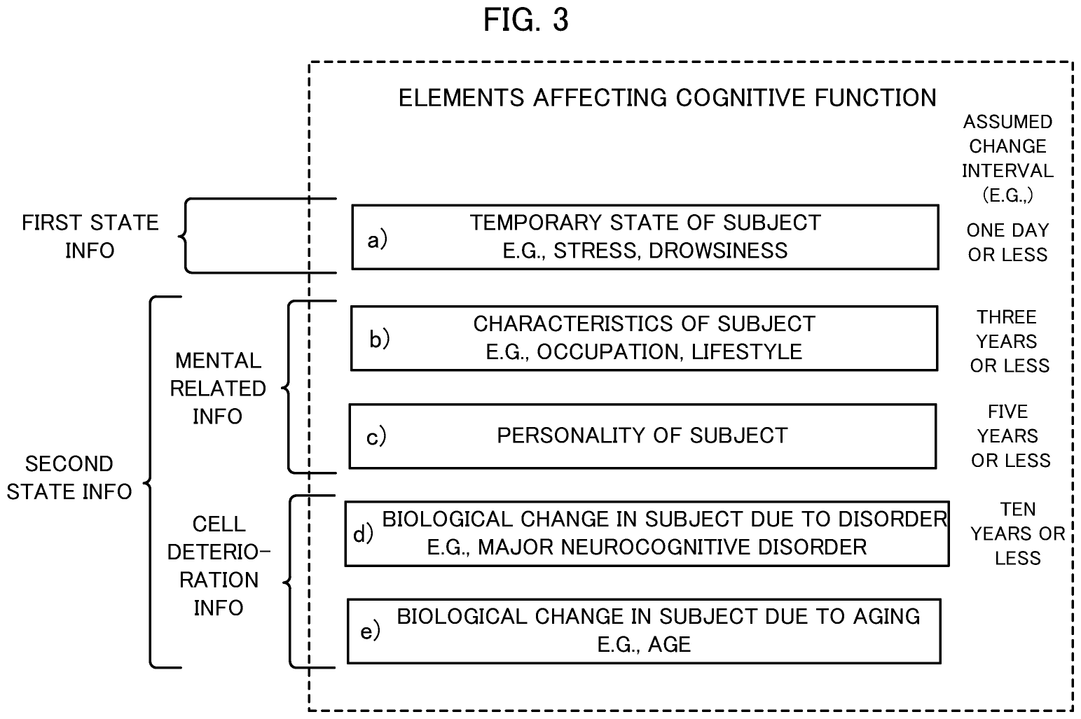

FIG. 3 is a diagram schematically illustrating elements affecting the cognitive function. As shown in FIG. 3, the cognitive function of the subject can be affected by
a) temporary state of the subject,
b) characteristics of the subject,
c) personality of the subject,
d) biological change in the subject due to disorder,
e) biological change in the subject due to aging.

The element “a) temporary state of the subject” represents a temporary (and short-term changing) state such as subject's stress state and drowsiness. Examples of the element “b) characteristics of the subject” include the occupation of the subject, the life habit thereof, hobbies thereof, and favorites thereof. The element “d) biological change in the subject due to disorder” represents the biological change based on the disorder (illness) affecting the cognitive function such as major neurocognitive disorder. The element “e) biological change in the subject due to aging” represent changes due to aging.

In addition, each of these elements a) to e) has a different change interval. Specifically, the element “a) temporary state of the subject” is a state that changes with a cycle period of approximately one day or less, and the element “b) characteristics of the subject” is a state that changes with a cycle period of approximately three years or less that is longer than that of the element “a) temporary state of the subject”. The element “c) personality of the subject” is a state that changes with a cycle period of less than five years that is longer than that of the element “b) characteristics of the subject”. The element “d) biological change in the subject due to disorder” is a state that changes with a cycle period of less than ten years that is longer than that of the element “c) personality of the subject”. The element “e) biological change in the subject due to aging” is an element which does not change by living environment of the subject, and in principle, changes according to age.

Then, the first state information is information regarding the element “a) temporary state of the subject”. It is noted that each of a stress state and drowsiness cited as an example of the element “a) temporary state of the subject” corresponds to a state or information to be estimated based on the first state information (e.g., facial data, gait data, voice data, and subjective questionnaire results regarding the subject) to be described later. The second state information is information regarding the elements “b) characteristics of the subject”, “c) personality of the subject”, “d) biological change in the subject due to disorder”, and “e) biological change in the subject due to aging”. Among the second state information, information regarding the elements “b) characteristics of the subject” and “c) personality of the subject” is information (referred to as “mental related information”) relating to a mental state of the subject and information which affects the subject's perceptions. Among the second state information, information regarding the elements “d) biological change in the subject due to disorder” and “e) biological change in the subject due to aging” is information (also referred to as “cell deterioration information”) regarding the degree (in other words, the degree of deterioration of the cells) of the basic physical health. The cell degradation information includes not only information regarding age and illness but also information regarding gender and race.

As described above, the cognitive function is affected by both the first state and the second state. Taking the above into consideration, the cognitive function estimation device 1 estimates the cognitive function of the subject with high accuracy by estimating the cognitive function of the subject based on the first state information and the second state information obtained from the measurement results of the subject.

Here, the cognitive function to be estimated will be supplementally described. For example, the cognitive function is divided into an intelligent function (including linguistic understanding, perceptual integration, working memory, processing speed), an attentional function, a frontal lobe function, a linguistic function, a memory function, a visual space cognitive function, and a directed attention function. Then, for example, the PVT task and WAIS-III are examples of a method of examining the intelligent function, and CAT (Clinical Assessment for Attention) is an example of a method of examining the attentional function, and the Trail marking test is an example of a method of examining the frontal lobe function. Besides, the WAB (Western Aphasia Battery) test and Category Fluency test are examples of a method of examining the linguistic function, and the Rey complex figure test is an example of a method of examining the visual space cognitive function, and BIT (Behavioral Inattention Test) is an example of a method of examining the directed attention function. These examinations are examples, and it is possible to measure the cognitive function by any other neuropsychological examinations. For example, there are testing methods such as N-back test and examination based on computational problems for a simple method of examining the cognitive function that can be conducted outside medical institutions.

(4) Functional Blocks

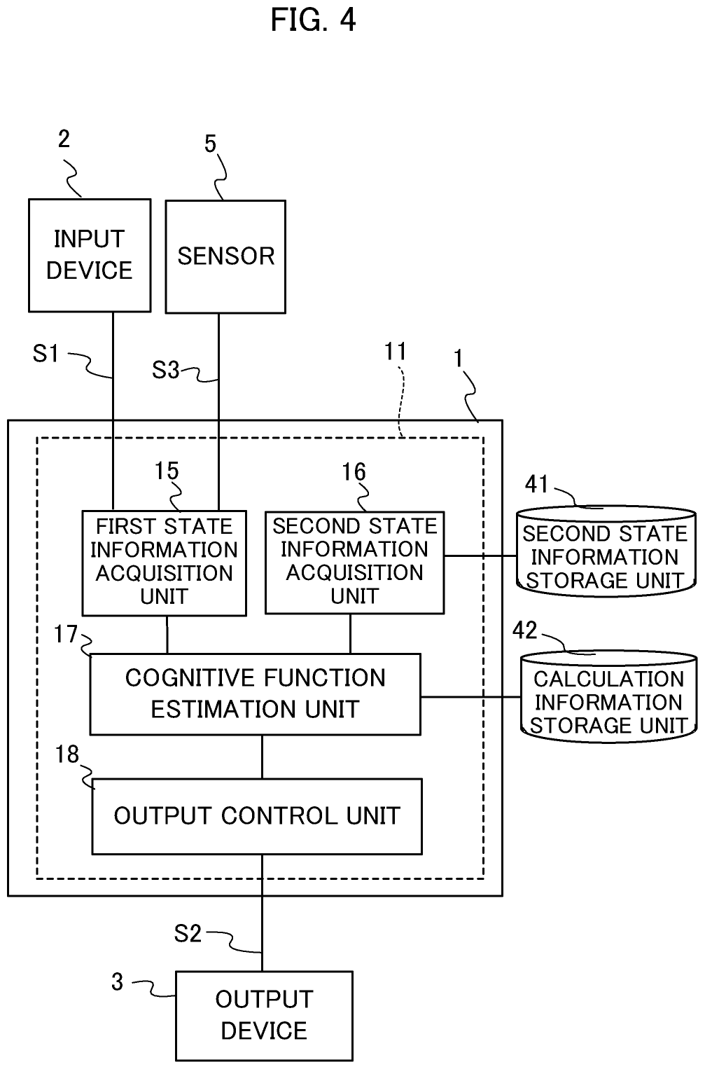

FIG. 4 is an example of functional blocks of the cognitive function estimation device 1. The processor 11 of the cognitive function estimation device 1 functionally includes a first state information acquisition unit 15, a second state information acquisition unit 16, a cognitive function estimation unit 17, and an output control unit 18. In FIG. 4, blocks to exchange data with each other are connected by a solid line, but the combination of blocks to exchange data with each other is not limited to FIG. 4. The same applies to the drawings of other functional blocks described below.

The first condition information acquisition unit 15 receives the input signal S1 supplied from the input device 2 and/or the sensor signal S3 supplied from the sensor 5 through the interface 13 and generates the first state information regarding the subject based on these signals. In this instance, the input signal S1 to be used for generating the first state information corresponds to the measurement information obtained by subjectively measuring the temporary state of the subject, and the sensor signal S3 to be used for generating the first state information corresponds to the measurement information obtained by objectively measuring the temporary state of the subject. The first state information acquisition unit 15 generates, as the first state information, facial data (e.g., video data showing a subject's face), gait data (e.g., video data showing a subject's walking), which is measurement information relating to the subject's gait state, voice data representing a voice uttered by the subject, or questionnaire results for subjectively measuring the degree of arousal, concentration, or tension of the subject.

In this case, for example, the first condition information acquisition unit 15 may generate the first state information that conforms to the input format of the inference model to be used by the cognitive function estimation unit 17. For example, the first state information acquisition unit performs a feature extraction process on the facial data, the gait data, the voice data, and/or the subjective questionnaire results described above. Then, the first state information acquisition unit 15 uses a tensor (e.g., feature vector) in a predetermined format obtained by the feature extraction process as the first state information. The above-mentioned feature extraction process may be a process based on any feature extraction technique (including the feature extraction technique using a neural network). The first state information acquisition unit 15 supplies the generated first state information to the cognitive function estimation unit 17.

When a questionnaire to the subject is conducted, the first state information acquisition unit 15 displays a screen image for answering the questionnaire on the output device 3 by transmitting the output signal S2, which is a display signal for displaying the screen image for answering the questionnaire, to the output device 3 via the interface 13. The first state information acquisition unit 15 receives the input signal S1 representing the response from the input device 2 through the interface 13.

The second state information acquisition unit 16 extracts the second state information regarding the subject from the second state information storage unit 41 and supplies the extracted second state information to the cognitive function estimation unit 17. The second condition information acquisition unit 16 may convert the second state information extracted from the second state information storage unit 41 so as to conform to the input format of the model to be used by the cognitive function estimation unit 17. In this case, the second condition information acquisition unit 16 performs feature extraction process to convert the second state information extracted from the second state information storage unit 41 into a tensor (e.g., a feature vector with a predetermined number of dimensions) in a predetermined format. The second state information after the conversion in the tensor format described above may be stored in advance in the second state information storage unit 41.

The cognitive function estimation unit 17 estimates the cognitive function of the subject based on the first state information supplied from the first state information acquisition unit 15, the second state information supplied from the second state information acquisition unit 16, and the calculation information stored in the calculation information storage unit 42. In this case, for example, the cognitive function estimation unit 17 calculates, based on the second state information, the estimated score of the cognitive function by correcting the temporal score of the cognitive function calculated based on the first state information. In another example, the cognitive function estimation unit 17 determines the estimated score of the cognitive function based on information outputted by an inference model built based on the calculation information, wherein the information is outputted by the inference model when the first state information and the second state information are inputted to the inference model. The cognitive function estimation unit 17 supplies the estimation result of the cognitive function of the subject to the output control unit 18.

The output control unit 18 outputs information relating to the estimation result of the cognitive function of the subject. For example, the output control unit 18 displays the estimation result of the cognitive function outputted by the cognitive function estimation unit 17 on the display unit of the output device 3 or outputs a sound (voice) by the sound output unit of the output device 3. In this case, for example, the output control unit 18 may compare the estimated result of the cognitive function with a reference value for determining the presence or absence of disorder of the cognitive function, and perform a predetermined notification to the subject or its manager based on the comparison result. For example, if the estimated result of the cognitive function is lower than the reference value, the output control unit 18 outputs information (warning information) prompting the person to go to a hospital or outputs advice information as to increase in the sleeping time. The output control unit 18 may acquire the contact information to contact the family of the subject from the storage device 4 or the like if the estimation result of the cognitive function falls below the above-described reference value and notify the subject's family of the information regarding the estimation result of the cognitive function.

Here, the above-described reference value may be a reference value determined based on time series estimation results of the cognitive function of the subject, or may be a general-purpose reference value for determining the presence or absence of cognitive disorder. In the former case, the cognitive function estimation unit 17 stores the estimation result of the cognitive function in the storage device 4 in association with the identification information of the subject, and the output control unit 18 sets the above-described reference value based on a statistical value (i.e., a representative value such as an average value and a median value) of the time series estimation results of the cognitive function of the subject stored in the storage device 4. In this case, the output control unit 18 may set the statistical value described above as a reference value, or may set a value lower than the statistical value described above by a predetermined value or a predetermined rate as the reference value. In the latter case, a general-purpose reference value for determining the presence or absence of cognitive disorder is stored in advance in the storage device 4 or the like, and the output control unit 18 acquires the general-purpose reference value and compares the general-purpose reference value with the estimated result of the cognitive function generated by the cognitive function estimation unit 17.

According to the configuration shown in FIG. 4, the cognitive function estimation device 1 can easily (i.e., easily without load of measurement) and accurately estimate the cognitive function of the subject based on the measurement by the sensor 5 or a simple input to the input device 2. Then, the cognitive function estimation device 1 outputs the estimation result of the cognitive function simply and accurately estimated as described above, thereby prompting subject's self-care and therefore promoting early detection or prevention of the cognitive function deterioration or the like.

Here, for example, each component of the first condition information acquisition unit 15, the second state information acquisition unit 16, the cognitive function estimation unit 17 and the output control unit 18 as described in FIG. 4 can be realized by the processor 11 executing a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology. The above explanation is also true for other embodiments to be described below.

(5) Specific Examples

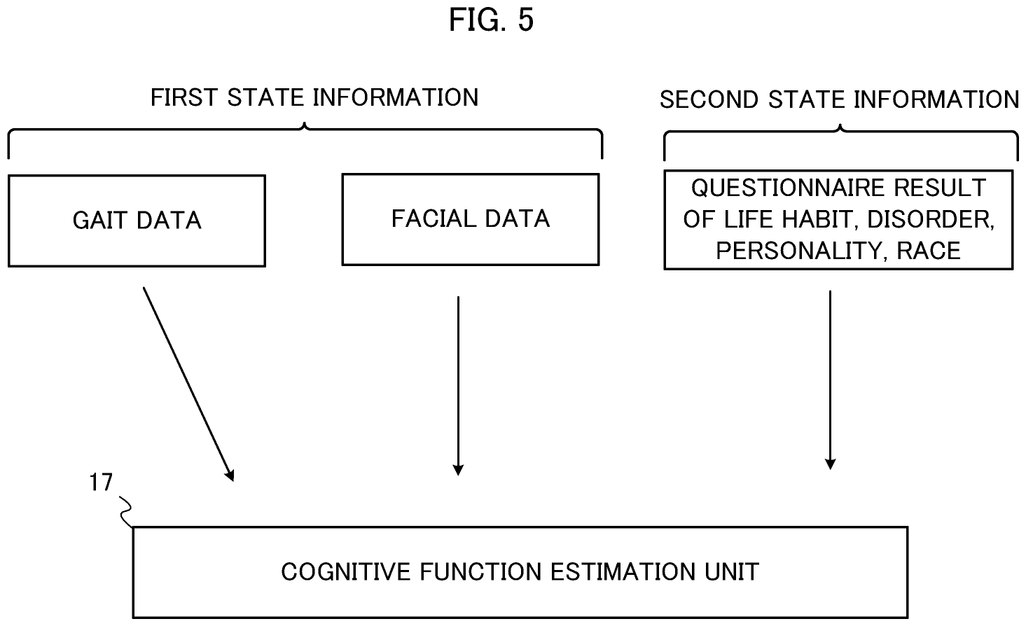

FIG. 5 is a diagram illustrating a specific example of estimation of the cognitive function. FIG. 5 shows an example estimation of cognitive function in which gait data and facial data are used as the first condition information and questionnaire results on life habit, disorder, personality, and race are used as the second state information.

In the example shown in FIG. 5, for example, the first state information acquisition unit 15 acquires the gait data and the facial data of the subject based on video data outputted by a camera included in the sensor 5 and supplies the acquired data to the cognitive function estimation unit 17. In this case, for example, the camera is provided at a position (including, for example, the residence or the workplace of the subject) where the subject can be photographed. Based on an image recognition technology, the first state information acquisition unit 15 extracts, as gait data, images in which subject's walking is displayed from video (time series images) outputted by the camera, and extracts, as facial data, an image in which the subject's face is displayed.

In addition, the questionnaire result, which is the second state information, is generated based on a questionnaire previously conducted, and is previously stored in the second state information storage unit 41, and the second state information acquisition unit 16 supplies the above-described questionnaire result stored in the second state information storage unit 41 to the cognitive function estimation unit 17. For example, the first condition information acquisition unit 15 and the second state information acquisition unit 16 convert the above-described respective information into tensors in a predetermined format by performing a predetermined feature extraction process, and supply the first state information and the second state information represented as the tensors in the predetermined format to the cognitive function estimation unit 17.

Then, with reference to the calculation information, the cognitive function estimation unit 17 estimates the cognitive function of the subject based on the gait data and the facial data of the subject obtained by the first state information acquisition unit 15 and the subject's questionnaire result regarding the life habit, disorder, personality, and race obtained by the second state information acquisition unit 16.

According to the example embodiment illustrated in FIG. 5, the cognitive function estimation device 1 acquires the sensor signal S3 outputted by a non-contact sensor (in this case, a camera or the like) and refers to the information stored in advance in the storage device 4. This enables the cognitive function estimation device 1 to estimate the cognitive function of the subject without giving an excessive measuring load to the subject. Then, the subject or the manager thereof can easily grasp the estimation result of the cognitive function based on the output information on the estimation result of the cognitive function outputted by the cognitive function estimation device 1. In addition, the cognitive function estimation device 1 estimates the cognitive function in a multi-angle manner by using, as the first state information, gait data related to the directed attention function which is one element of the cognitive function and facial data related to the attentional function which is another element of the cognitive function. This enables the cognitive function estimation device 1 to estimate the cognitive function with high accuracy while estimating a wide range of functions in the cognitive function. In other words, the cognitive function estimation device 1 estimates the cognitive function in a multilateral manner by considering a plurality of elements such as the attentional function and the directed attention function among cognitive functions, thereby estimating wide-ranging functions with high accuracy.

In addition, the cognitive function estimation device 1 estimates the cognitive function using the second state information indicating: the life habit such as lack of motion which affects gait (see “b) characteristics of subject” in FIG. 3); disorder such as injury of the foot and; the personality and race which affects facial expression. Thus, the cognitive function estimation device 1 can obtain an accurate estimation result of the cognitive function by accurately considering the second state related to (affecting) the first state.

The cognitive function estimation device 1 may estimate the cognitive function using the voice data of the subject as the first state information in addition to the gait data and the facial data. In this case, the sensor 5 includes a voice input device, and supplies voice data generated when a subject utters to the cognitive function estimation device 1, and the first state information acquisition unit 15 of the cognitive function estimation device 1 acquires the voice data as a part of the first state information. According to this embodiment, the cognitive function estimation device 1 can estimate the cognitive function more comprehensively by using the voice data related to the linguistic function which is an element of the cognitive function different from the elements of the cognitive function related to gait data and facial data. In addition, even in this case, the cognitive function estimation device 1 can easily estimate the cognitive function of the subject based on the output from a non-contact sensor (voice input device) without increasing the load of measurement.

Next, a supplementary description will be given of technical effects in the specific example shown in FIG. 5. In general, it is said that the decrease in cognitive function is related to the decrease in walking speed and the decrease in the rotation angle of the foot. On the other hand, even when the walking speed is judged to be slower than the reference speed and/or the rotation angle of the foot is judged to be smaller than the reference angle based on the gait data, it is not distinguished whether it is caused by the deterioration of the cognitive function of the subject, it is caused by the life habit of insufficient exercise, or it is caused by the injury (disorder) of the foot. Therefore, when the cognitive function is estimated without considering the life habit or the disorder, there is a possibility that the estimated score of the cognitive function of the subject who habitually lacks exercise or the subject having the injury (disorder) of the foot could become excessively low value and therefore it could be determined that there is an abnormality in the cognitive function of a subject who has a normal cognitive function.

Similarly, it is generally said that the decrease in the cognitive function is related to the decrease in the movement of facial expression. On the other hand, even when the movement of the facial expression is judged to be smaller than the reference value based on the facial data, it is difficult to distinguish whether it is caused by the deterioration of the cognitive function of the subject, or it is caused by the personality of the subject (personality which tends to harden the facial expression) or it is caused by the race (race which tends to have a small facial expression) of the subject. Therefore, when the cognitive function is estimated without considering the personality and/or race, the estimated score of the cognitive function of the subject with a personality which tends to harden the facial expression or with a race which tends to have a small facial expression tends to be small. Therefore, in this case, there is a possibility that the cognitive function could be determined to be abnormal even for a subject who has a normal cognitive function.

Taking the above into consideration, in the specific example shown in FIG. 5, the cognitive function estimation device 1 estimates the cognitive function of the subject in consideration of the second state information (the questionnaire result of life habit, disorder, personality, and race) which is related to the first state information (gait data, facial data). Thus, the cognitive function estimation device 1 can obtain an accurate estimation result of the cognitive function.

(6) Learning of Inference Model

Next, a description will be given of a method of learning an inference model (i.e., a method of generating calculation information) in such a case, as an example, that a trained inference model is used in the estimation of the cognitive function. Hereafter, as an example, a case in which the cognitive function estimation device 1 performs learning of the inference model will be described, but a device other than the cognitive function estimation device 1 may perform the learning of the inference model.

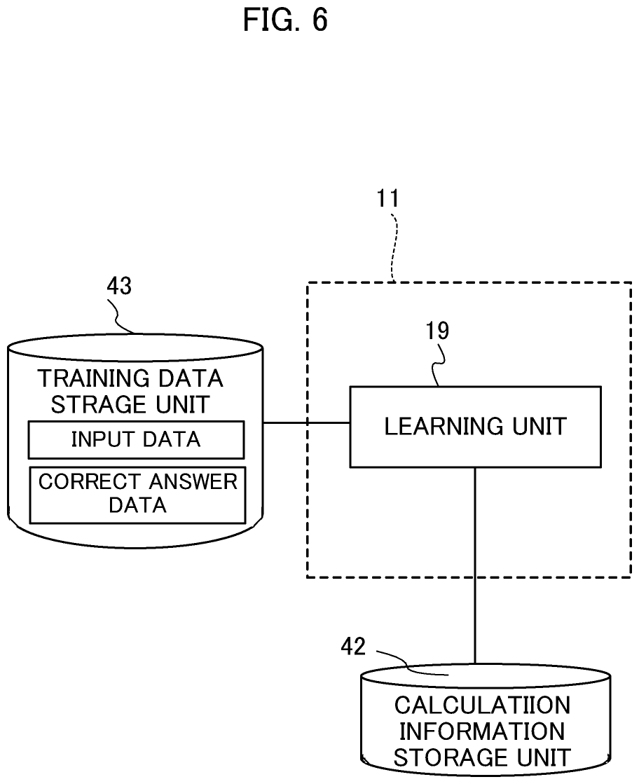

FIG. 6 is an example of functional blocks of the processor 11 of the cognitive function estimation device 1 relating to the learning of the inference model. Regarding the learning of the inference model, the processor 11 functionally includes a learning unit 19. The storage device 4 further includes a training data storage unit 43. The training data storage unit 43 stores training data including input data and correct answer data. The input data is data to be inputted to the inference model in training the inference model, and the correct data indicates a correct answer of the estimation result (i.e., correct score) of the cognitive function to be outputted by the inference model when the input data described above is inputted to the inference model in training the inference model.

Here, the input data includes the first state information and the second state information. In this instance, the first state information is data generated by applying the same process as the process that is executed by the first state information acquisition unit 15 to data (i.e., data equivalent to the input signal S1 and the sensor signal S3 in FIG. 1 and FIG. 4) for training that is measured subjectively or objectively from the subject or a person other than the subject. The second state information included in the input data may be the same data as the second state information stored in the second state information storage unit 41 or may be data separately generated for training.

It is noted that the input data is represented, through a feature extraction process already referred to in the description related to FIG. 4, as a tensor in a predetermined format to conform to the input format of the inference model, for example. Such a feature extraction process may be executed by a device that performs the learning (the cognitive function estimation device 1 in the case of the example shown in FIG. 6).

The correct answer data is, for example, a diagnosis result regarding the cognitive function of the subject or a person other than the subject or an examination result of a neuropsychological examination of the cognitive function. Specifically, the examination results based on various examination (test) methods related to the cognitive function described in the section “(3) Specific Examples of First State and Second State” are adopted as the correct answer data.

At a stage before the estimation processing of the cognitive function, the learning unit 19 performs learning for generating the calculation information that is parameters of the inference model to be stored in the calculation information storage unit 42 with reference to the training data storage unit 43. In this case, for example, the learning unit 19 determines the parameters of the inference model such that the error (loss) between the information outputted by the inference model when the input data is inputted to the inference model and the correct answer data corresponding to the input data that is inputted is minimized. The algorithm for determining the parameters to minimize the error may be any learning algorithm used in machine learning, such as the gradient descent method and the error back propagation method. Then, the learning unit 19 stores the parameters of the inference model after the training in the training data storage unit 43 as the calculation information.

(7) Processing Flow

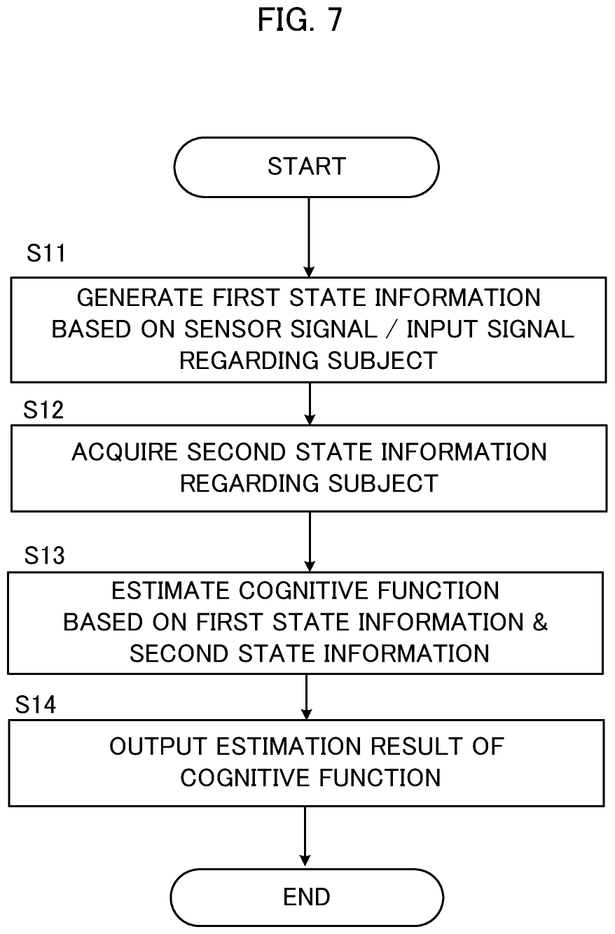

FIG. 7 is an example of a flowchart illustrating a processing procedure of the cognitive function estimation device 1 related to the estimation of the cognitive function. For example, the cognitive function estimation device 1 determines that it is the timing of estimating the cognitive function to execute the process of the flowchart shown in FIG. 7, if a predetermined execution condition of estimating the cognitive function is satisfied. The cognitive function estimation device 1 determines that the above-described execution condition of the estimation is satisfied, for example, if the input device 2 receives an input signal S1 instructing the execution of the estimation process of the cognitive function. In addition, the cognitive function estimation device 1 may refer to the execution condition of the estimation stored in advance in the storage device 4 or the like and determine whether or not the execution condition of the estimation is satisfied. In another case, it may determine that the execution condition of the estimation is satisfied if it is on a predetermined date and time set in advance (for example, a predetermined time of every day). In yet another example, the cognitive function estimation device 1 may determine that the execution condition of the estimation has been met when it acquires the sensor signal S3 and/or the input signal S1 enough to generate the first state information that is required for estimation of the cognitive function.

First, the first state information acquisition unit 15 of the cognitive function estimation device 1 generates the first state information based on the sensor signal S3 and/or the input signal S1 that are measured information regarding the subject at the above-described timing of estimating the cognitive function (step S11). In this instance, the first state information acquisition unit 15 acquires the sensor signal S3 indicating the objective measurement information regarding the subject from the sensor 5 and/or the input signal S1 indicating the subjective measurement information regarding the subject from the input device 2 through the interface 13, and generates the first state information based on the acquired signal. In this case, for example, the first state information acquisition unit 15 may perform a predetermined feature extracting process on the acquired sensor signal S3 or/and the input signal S1 thereby to generate the first state information which conforms to the input format of the model to be used by the cognitive function estimation unit 17.

The second condition information acquisition unit 16 of the cognitive function estimation device 1 acquires the second state information of the subject (step S12). In this case, the second condition information acquisition unit 16 acquires the second state information of the subject from the second state information storage unit 41 via the interface 13. For example, the second state information acquisition unit 16 may perform a predetermined feature extraction process on the information extracted from the second state information storage unit 41 to generate the second state information which conforms to the input format of the model used by the cognitive function estimation unit 17.

Next, the cognitive function estimation unit 17 of the cognitive function estimation device 1 estimates the cognitive function of the subject based on the first state information acquired at step S11 and the second state information acquired at step S12 (step S13). In this case, the cognitive function estimation unit 17 acquires the estimation result of the cognitive function outputted by the inference model by inputting the first state information and the second state information into the inference model built based on the calculation information stored in the calculation information storage unit 42, for example. The above-described inference model may be a learning model, as described above, or may be an expression or a look-up table or the like.

Then, the output control unit 18 of the cognitive function estimation device 1 outputs information relating to the estimation result of the cognitive function calculated at step S13 (step S14). In this instance, the output control unit 18 supplies the output signal S2 to the output device 3 so that the output device 3 performs a display or audio output representing the estimated result of the cognitive function. In this case, for example, the output control unit 18 compares the estimation result of the cognitive function with a predetermined reference value, and based on the comparison result, notifies the subject or the manager of the subject of information regarding the estimation result of the cognitive function. Thus, the cognitive function estimation device 1 can suitably present information regarding the estimation result of the cognitive function of the subject to the subject or the manager thereof.

(8) Modification

The cognitive function estimation device 1 may estimate the cognitive function of the subject based on the first state information without using the second state information.

In this case, the cognitive function estimation device 1 estimates the cognitive function of the subject based on the gait data and the facial data in the example shown in FIG. 5, for example. In this case, for example, the calculation information stored in the calculation information storage unit 42 includes parameters of the inference model configured to output the estimation result of the cognitive function when the first state information is inputted to the inference model, and the output control unit 18 estimates the cognitive function of the subject from the first state information by using the inference model built based on the calculation information. In this modification, the storage device 4 does not need to have a second state information storage unit 41.

According to this modification, the cognitive function estimation device 1 acquires the gait data relating to the directed attention function and the facial data relating to the attentional function based on the output from a non-contact sensor (in this case, a camera or the like). This enables the cognitive function estimation device 1 to estimate the cognitive function with high accuracy without giving a measurement load to the subject while estimating a wide range of functions in the cognitive function. In other words, the cognitive function estimation device 1 estimates the cognitive function in a multilateral manner by considering a plurality of elements such as the attentional function and a directed attention function among elements of the cognitive function, thereby estimating the various functions with high accuracy.

### Second Example Embodiment

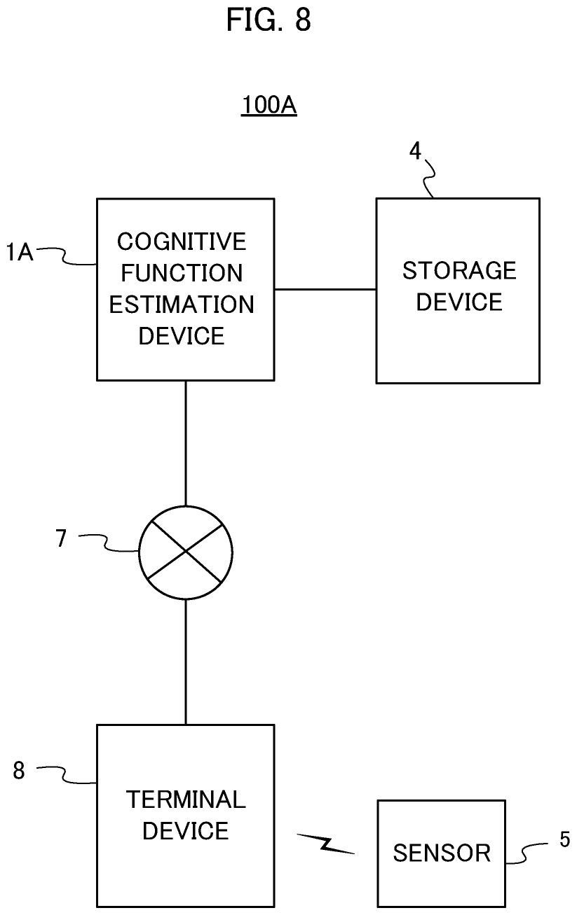

FIG. 8 shows a schematic configuration of a cognitive function estimation 100A according to a second example embodiment. The cognitive function estimation system 100A according to the second example embodiment is a system which conforms to a server-client model, and a cognitive function estimation device 1A functioning as a server device performs a process of the cognitive function estimation device 1 in the first example embodiment. Hereinafter, the same components as those in the first example embodiment are appropriately denoted by the same reference numerals, and a description thereof will be omitted.

As shown in FIG. 8, the cognitive function estimation system 100A mainly includes a cognitive function estimation device 1A that functions as a server, a storage device 4 that stores substantially the same data as in the first example embodiment, and a terminal device 8 that functions as a client. The cognitive function estimation device 1A and the terminal device 8 perform data communication with each other via the network 7.

The terminal device 8 is a terminal having an input function, a display function, and a communication function, and functions as the input device 2 and the output device 3 shown in FIG. 1. The terminal device 8 may be, for example, a personal computer, a tablet-type terminal, or a PDA (Personal Digital Assistant), or the like. The terminal device 8 transmits a biological signal outputted by one or more sensors (not shown) or an input signal based on a user input to the cognitive function estimation device 1A.

The cognitive function estimation device 1A has the same configuration as the cognitive function estimation device 1 shown in FIGS. 1, 2, and 4, for example. Then, the cognitive function estimation device 1A receives information, which is information obtained by the cognitive function estimation device 1 shown in FIG. 1 through the input device 2 and the sensor 5, from the terminal device 8 via the network 7, and estimates the cognitive function of the subject based on the received information. In addition, the cognitive function estimation device 1A transmits an output signal indicating the information regarding the above-described estimation result to the terminal device 8 through the network 7, in response to a request from the terminal device 8. Namely, in this case, the terminal device 8 functions as the output device 3 in the first example embodiment. Thus, the cognitive function estimation device 1A suitably presents information regarding the estimation result of the cognitive function to the user of the terminal device 8.

### Third Example Embodiment

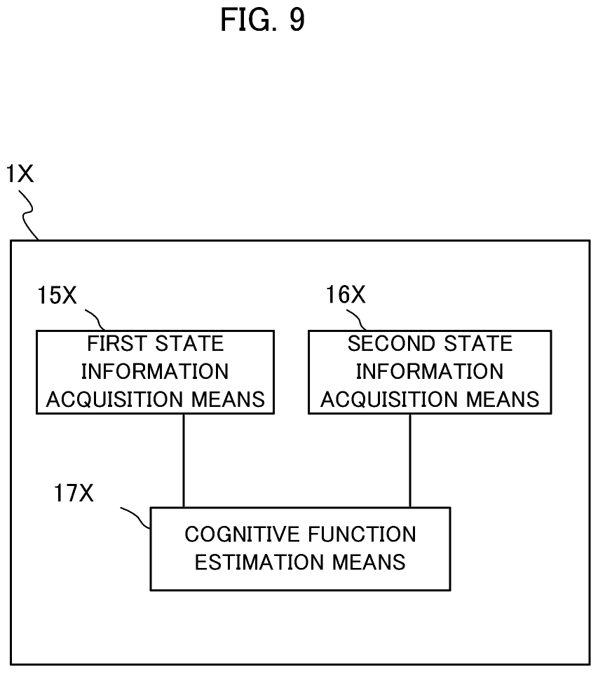

FIG. 9 is a block diagram of the cognitive function estimation device 1X according to the third example embodiment. The cognitive function estimation device 1X mainly includes a first state information acquisition means 15X, a second state information acquisition means 16X, and a cognitive function estimation means 17X. The cognitive function estimation device 1X may be configured by a plurality of devices.

The first state information acquisition means 15X is configured to acquire first state information representing a first state of a subject regarding a cognitive function of the subject. Examples of the first state information acquisition means 15X include the first state information acquisition unit 15 in the first example embodiment or the second example embodiment.

The second state information acquisition means 16X is configured to acquire second state information representing a second state of the subject whose interval (not necessarily constant cycle period, hereinafter the same) of state change is longer than the first state. Examples of the second condition information acquisition means 16X may be the second state information acquisition unit 16 in the first example embodiment (excluding the modification, hereinafter the same in the third example embodiment) or the second example embodiment.

The cognitive function estimation means 17X is configured to estimate the cognitive function of the subject based on the first state information and the second state information. The cognitive function estimation unit 17X may be, for example, the cognitive function estimation unit 17 in the first example embodiment or the second example embodiment.

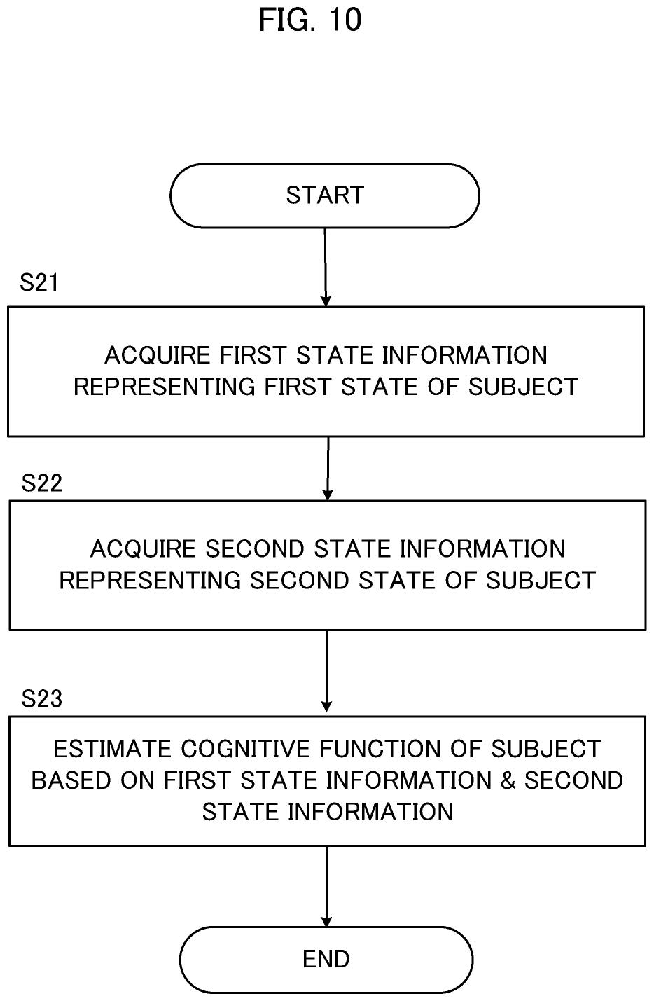

FIG. 10 is an exemplary flowchart that is executed by the cognitive function estimation device 1X in the third example embodiment. The first state information acquisition means 15X acquires first state information representing a first state of a subject regarding a cognitive function of the subject (step S21). The second state information acquisition means 16X is configured to acquire second state information representing a second state of the subject whose interval of state change is longer than the first state (step S22). The cognitive function estimation means 17X estimates the cognitive function of the subject based on the first state information and the second state information (step S23).

According to the third example embodiment, the cognitive function estimation device 1X can accurately estimate the cognitive function of the subject.

### Fourth Example Embodiment

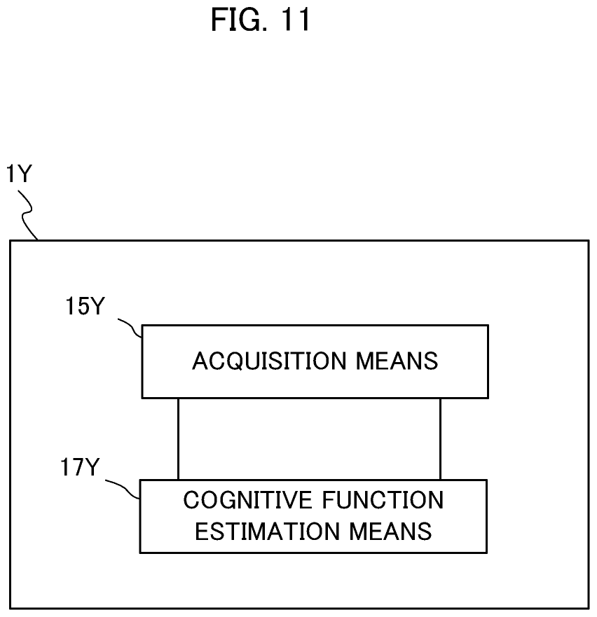

FIG. 11 is a block diagram of the cognitive function estimation device 1Y according to the fourth example embodiment. The cognitive function estimation device 1Y mainly includes an acquisition means 15Y and a cognitive function estimation means 17Y The cognitive function estimation device 1Y may be configured by a plurality of devices.

The acquisition means 15Y is configured to acquire facial data which is measurement information regarding a face of a subject and gait data which is the measurement information regarding a gait state of the subject. Examples of the acquisition means 15Y includes the first state information acquisition unit 15 in the first example embodiment (including the modification) or the second example embodiment.

The cognitive function estimation mean 17Y is configured to estimate a cognitive function of the subject based on the facial data and the gait data. Examples of the cognitive function estimation means 17Y include the cognitive function estimation unit 17 in the first example embodiment (including the modification) or the second example embodiment.

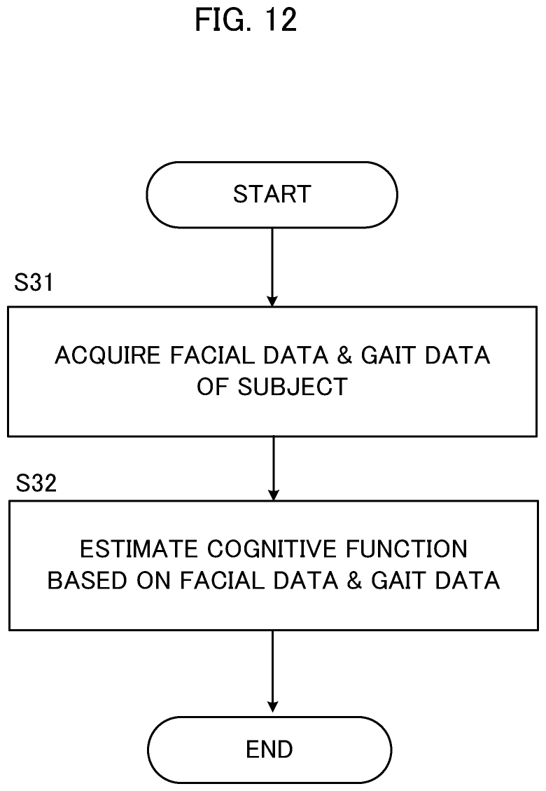

FIG. 12 is an exemplary flowchart that is executed by the cognitive function estimation device 1Y in the fourth example embodiment. The acquisition means 15Y acquires facial data which is measurement information regarding a face of a subject and gait data which is the measurement information regarding a gait state of the subject (step S31). The cognitive function estimation mean 17Y estimates a cognitive function of the subject based on the facial data and the gait data (step S32).

The cognitive function estimation device 1X according to the fourth example embodiment can estimate the cognitive function of the subject with high accuracy without giving excessive load of measurement to the subject.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

### [Supplementary Note 1]

A cognitive function estimation device comprising:
a first state information acquisition means configured to acquire first state information representing a first state of a subject regarding a cognitive function of the subject;
a second state information acquisition means configured to acquire second state information representing a second state of the subject whose interval of state change is longer than the first state; and
a cognitive function estimation means configured to estimate the cognitive function of the subject based on the first state information and the second state information.

### [Supplementary Note 2]

The cognitive function estimation device according to Supplementary Note 1,
wherein the second state information acquisition means is configured to acquire the second state information representing the second state related to the first state.

### [Supplementary Note 3]

The cognitive function estimation device according to Supplementary Note 1 or 2,
wherein the second state information acquisition means is configured to acquire the second state information including mental related information which is information related to a mental state of the subject.

### [Supplementary Note 4]

The cognitive function estimation device according to Supplementary Note 3,
wherein the mental related information is information regarding at least one of a personality, an occupation, a hobby, a preference, and a life habit of the subject.

### [Supplementary Note 5]

The cognitive function estimation device according to any one of Supplementary Notes 1 to 4,
wherein the second state information acquisition means is configured to acquire the second state information including cell deterioration information that is information regarding a degree of deterioration of cells of the subject.

### [Supplementary Note 6]

The cognitive function estimation device according to any one of Supplementary Notes 1 to 5,
wherein the first state information acquisition means is configured to acquire the first state information including
   facial data which is measurement information regarding a face of the subject and
   gait data which is measurement information regarding a gait state of the subject.

### [Supplementary Note 7]

The cognitive function estimation device according to Supplementary Note 6,
wherein the first state information acquisition means is configured to acquire the first state information further including
   voice data which is measurement information regarding a voice of the subject.

### [Supplementary Note 8]

The cognitive function estimation device according to any one of Supplementary Notes 1 to 7,
wherein the first state information acquisition means is configured to generate the first state information based on the subjectively or objectively measured information from the subject at an estimation timing of the cognitive function, and
wherein the second state information acquisition means is configured to acquire the second state information from a storage device storing the second state information.

### [Supplementary Note 9]

The cognitive function estimation device according to any one of Supplementary Notes 1 to 8, further comprising
an output control means configured to output information regarding an estimation result of the cognitive function.

### [Supplementary Note 10]

A cognitive function estimation device comprising:
an acquisition means configured to acquire
   facial data which is measurement information regarding a face of a subject and
   gait data which is measurement information regarding a gait state of the subject; and
a cognitive function estimation mean configured to estimate a cognitive function of the subject based on the facial data and the gait data.

### [Supplementary Note 11]

A cognitive function estimation method executed by a computer, the cognitive function estimation method comprising:
acquiring first state information representing a first state of a subject regarding a cognitive function of the subject;
acquiring second state information representing a second state of the subject whose interval of state change is longer than the first state; and
estimating the cognitive function of the subject based on the first state information and the second state information.

### [Supplementary Note 12]

A cognitive function estimation method executed by a computer, the cognitive function estimation method comprising:
acquiring
   facial data which is measurement information regarding a face of a subject and
   gait data which is measurement information regarding a gait state of the subject; and
estimating a cognitive function of the subject based on the facial data and the gait data.

### [Supplementary Note 13]

A storage medium storing a program executed by a computer, the program causing the computer to
acquire first state information representing a first state of a subject regarding a cognitive function of the subject;
acquire second state information representing a second state of the subject whose interval of state change is longer than the first state; and
estimate the cognitive function of the subject based on the first state information and the second state information.

### [Supplementary Note 14]

A storage medium storing a program executed by a computer, the program causing the computer to
acquire
   facial data which is measurement information regarding a face of a subject and
   gait data which is measurement information regarding a gait state of the subject; and
estimate a cognitive function of the subject based on the facial data and the gait data.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### INDUSTRIAL APPLICABILITY

Examples of the applications include a service related to management (including self-management) to grasp and maintain the cognitive function.

### DESCRIPTION OF REFERENCE NUMERALS

1, 1A, 1X Cognitive function estimation device
2 Input device
3 Output device
4 Storage device
5 Sensor
8 Terminal device
100, 100A Cognitive function estimation system

1. A device for estimating cognitive function based on facial image data, the device comprising:
at least one memory storing instructions; and
at least one processor configured to execute the instructions to:
acquire first state information representing a measurement result of a temporary state of a subject regarding a cognitive function of the subject, the first state information including the facial image data and gate data, the facial image data being measurement information regarding a face of the subject, and the gait data being measurement information regarding a gait state of the subject;
acquire second state information representing a non-temporary state of the subject, the second state information determined based on the measurement result of the temporary state of the subject for a predetermined period; and
calculate a score of the cognitive function of the subject based on the first state information and the second state information.

2. The device according to claim 1,
wherein second state is related to the temporary state.

3. The device according to claim 1,
wherein the second state information includes mental related information related to a mental state of the subject.

4. The device according to claim 3,
wherein the mental related information is information regarding at least one of a personality, an occupation, a hobby, a preference, and a life habit of the subject.

5. The device according to claim 1,
wherein the second state information includes cell deterioration information regarding a degree of deterioration of cells of the subject.

6. (canceled)

7. The device according to claim 1,
wherein the first state information further includes voice data which is measurement information regarding a voice of the subject.

8. The device according to claim 1,
wherein the at least one processor is configured to execute the instructions to generate the first state information based on subjectively or objectively measured information from the subject at an estimation timing of the cognitive function, and
wherein the at least one processor is configured to execute the instructions to acquire the second state information from a storage device storing the second state information.

9. The device according to claim 1,
wherein the at least one processor is configured to further execute the instructions to output information regarding an estimation result of the cognitive function.

10. (canceled)

11. A method for estimating cognitive function based on facial image data, the method comprising:
acquiring, by a processor, first state information representing a measurement result of a temporary state of a subject regarding a cognitive function of the subject, the first state information including the facial image data and gate data, the facial image data being measurement information regarding a face of the subject, and the gait data being measurement information regarding a gait state of the subject;
acquiring, by the processor, second state information representing a non-temporary state of the subject, the second state information determined based on the measurement result of the temporary state of the subject for a predetermined period; and
calculating, by the processor, a score of the cognitive function of the subject based on the first state information and the second state information.

12. (canceled)

13. A non-transitory computer readable storage medium storing a program executable by a computer to cause the computer to:
acquire first state information representing a measurement result of a temporary state of a subject regarding a cognitive function of the subject, the first state information including the facial image data and gate data, the facial image data being measurement information regarding a face of the subject, and the gait data being measurement information regarding a gait state of the subject;
acquire second state information representing a non-temporary state of the subject, the second state information determined based on the measurement result of the temporary state of the subject for a predetermined period; and
calculate a score of the cognitive function of the subject based on the first state information and the second state information.

14. (canceled)

15. The device according to claim 1,
wherein the at least one processor is configured to execute the instructions to
calculate the score of the cognitive function by inputting the acquired first state information and the acquired second state information into an estimation model, the estimation model having learned a relationship between the first state information, the second state information, and the score of the cognitive function by machine learning.

16. The device according to claim 9,
wherein the at least one processor is configured to execute the instructions to further output warning information or advice information for optimizing an activity of the subject, if the score of the cognitive function of the subject is lower than a predetermined value.
